(19) United States
(12) Patent Application Publication
(10) Pub. No.: US 2016/0101161 A1
(43) Pub. Date: Apr. 14, 2016

(54) ANTI-VIRAL THERAPEUTIC FOR INFECTION OF THE EYE

(71) Applicant: ANSUN BIOPHARMA, INC., San Diego, CA (US)

(72) Inventor: Ronald D. Moss, Encinitas, CA (US)

(21) Appl. No.: 14/893,226

(22) PCT Filed: Jun. 3, 2014
(86) PCT No.: PCT/US14/40719

§ 371 Date: Nov. 23, 2015

Related U.S. Application Data
(60) Provisional application No. 61/830,590, filed on Jun. 3, 2013.

(51) Int. Cl. A61K 38/47; A61K 38/18; A61K 9/00

(52) U.S. Cl. CPC A61K 38/47; A61K 9/0048; A61K 38/1808; C12Y 302/01018

(57) ABSTRACT

The present disclosure provides novel compositions and methods for treating an infection of the eye resulting from an infection of a member of the Picornavirdae virus family. In particular, the present disclosure provides compounds having an anchoring domain that anchors the compound to the surface of a target cell, and a sialidase domain that can act extracellularly to inhibit infection of a target cell by a pathogen, such as a virus. The present disclosure also comprises therapeutic compositions having sialidase activity, including protein based compounds having sialidase catalytic domains. Compounds of the disclosure can be used for treating pathogenic infection to the eye.

### RELATED APPLICATIONS

This application claims priority to U.S. Provisional Patent Application No. 61/830,590, filed on Jun. 3, 2013, the entire contents of which are hereby incorporated by reference.

### BACKGROUND

Acute hemorrhagic conjunctivitis (AHC) is a highly contagious viral syndrome. It is characterized by conjunctivitis, the inflammation of the outer most layer of the eye and the inner surface of the eyelid collectively referred to as the conjunctiva, as well as keratitis (inflammation of the cornea) foreign body sensation within the eye, and pain. Respiratory symptoms and severe neurological symptoms have also been associated with AHC (Bahri et al. J. Med. Microbiol. 54: 63-69; Palacios et al. J. Neurovirol. 11: 424-433). Epiphora, photophobia as well as subconjunctival hemorrhage, and congestion are also common symptoms of AHC. Clinical symptoms appear 12-48 hours following infection and remain for 1-2 weeks (Ghendon et al. (1989) University of Tokyo Press. 3-9).

AHC is a rapidly progressive viral infection and frequently causes outbreaks. The rapidly dispersing viral infection was first reported in Ghana in 1969 (Metselaar et al. (1976) Trop Geor Med. 28: 131-136; Chatterjee et al. (1970) Br. J. Opthalmol. 54: 628-630) and since, many outbreaks and three pandemics have taken place worldwide: 1969, 1980 and as recently as 2002-2004 (Mirkovic et al. (1973) Bull. W. H. O. 49: 341-346; Kew et al. (1983) Infect. Immun. 41: 631-635; Ghazali et al. (2003) Singapore Med. 44: 511-516). These epidemic outbreaks of AHC have occurred throughout the world in recent years. In 2002, an AHC pandemic began in the eastern hemisphere and within three months, left reportedly over one million people affected with the eye infection (Oh et al. (2003) Emerg. Infect. Dis. 9: 1010-1012). The following year, numerous outbreaks were reported worldwide including in South Korea (Park et al. J. Med. Virol. 78: 91-97), India (Gopalkrishna et al. J. Med. Virol. (2007) 79: 748-753), Tunisia (Triki et al. Clin. Microbiol. Infect. 13: 176-182.), and Congo (Leveque et al. Eur. J. Clin. Microbiol. Infect. Dis. 26: 199-202.) as well as in the western hemisphere, namely in Nicaragua, Honduras, Guatemala, El Salvador, in the Caribbean (Pan American Health Organization (2003)), Brazil (Maoura et al. Br. J. Opthalmol. 90: 1091-1093) and Puerto Rico (Alsonso-Eschanove et al. (2004) Morb. Mortal. Wkly. Rep. 53: 632-634).

AHC is caused by two members of the Picornavirdae family, coxsackievirus A24 variant (CVA24v) and enterovirus 70 (EV70). Picornavirdae viruses are positive-sense ssRNA viruses. AHC was initially characterized as being caused by EV70 in 1969; however AHC is more commonly the result of CVA24v. The majority of the outbreaks of AHC occurring in the past 15-20 years were caused by CVA24v. Since the identification of these causative agents, about 100 million cases of AHC have been estimated to have occurred. Coxsackievirus is a member of the genus Enterovirus which also includes the poliovirus, echovirus and rhinovirus. Coxsackieviruses are categorized into groups A and B: coxsackieviruses in group A infect the skin and mucous layers causing hand, foot and mouth disease, herpangina, and hemorrhagic conjunctivitis and those in group B trigger inflammatory heart muscle disease (myocarditis), pancreatitis and aseptic meningitis.

Coxsackieviruses are self-limiting and hence there is no specific treatment for their infections. However, due to the highly contagious manner of coxsackievirus through AHC, transmission via contact with infected persons or with contaminated objects is extremely highly. Further, although the symptoms may clear from an infected person in a matter of 1-2 weeks, the person may shed virus, and hence be contagious, for weeks. Further, rare but serious complications arise from coxsackievirus infection including infection of the heart and brain.

Treatment of this virus would be essential for controlling epidemics of the infection. As there is no vaccination or treatment for CVA24v, a novel therapeutic is essential to address future AHC pandemics. Attempts are currently underway to address the lack of treatment with small interfering RNA against these viruses which cause AHC by inhibition of viral replication (Jun et al. (2011) Inv. Opthamol. Vis. Sci. 52: 58-63).

### SUMMARY

The present disclosure provides new compositions and methods for treating AHC infection. Specifically, it provides compounds which can act extracellularly to reduce or prevent infection of a cell by a pathogen, e.g., CVA24v. Some preferred embodiments of the disclosure include therapeutic compounds having an anchoring domain that facilitates association of the compound with the surface of a target cell and a sialidase domain that can act extracellularly to reduce or prevent infection of the target cell by a pathogen, such as a virus. In some embodiments the compound comprises, consists of or consists essentially all or a catalytically active portion of a sialidase.

In one aspect, the disclosure provides a method for treating infection by a pathogen. In preferred embodiments, the method comprises administering a compound having sialidase activity, such as a sialidase or a fragment thereof containing a sialidase catalytic domain, including a sialidase catalytic domain fusion protein, to a subject to treat an infection. A pathogen can be, for example, a viral pathogen. The method includes administering a pharmaceutically effective amount of a compound of the present disclosure to at least one target cell of a subject. Preferably, the pharmaceutical composition can be administered by the use of eyedrops or other topical formulation.

The method of treatment is described for an infection of the eye caused by a virus. In a preferred embodiment, the virus is a member of the Picornavirdae family. In particular, the virus is a member of the genus Enterovirus. In a preferred embodiment, the virus is a coxsackievirus and an enterovirus. In a further preferred embodiment, the pathogen is CVA24v and EV70.

The infection of the eye can be associated with another infection. In some instances, the eye infection is associated with an infection of the upper respiratory tract. In another instance, the eye infection is associated with an infection of the throat. In another instance, the infection of the eye is associated with the common cold.

The method of treatment disclosed is for an infection of the eye. In a preferred embodiment, the infection is of the epithelial cells of the eye. In another embodiment, the infection is in the conjunctiva cells of the eye. In another embodiment, the infection is in the cornea cells of the eye.

In some cases the compound includes a glycosaminoglacan (GAG) binding domain. The GAG binding domain can be all or a fragment of: human platelet factor 4, human interleukin 8, human antithrombin III, human apoprotein E, human angio associated migratory protein, or human amphiregulin.

The source of the sialidase activity can be bacterial or human. In preferred embodiments, the bacterial source of the sialidase is selected from Vibrio cholera, Arthrobacter ureafaciens, Clostridium perfringens, Actinomyces viscosus, and Micromonospora viridifaciens.

In some cases, the compound having sialidase activity is DAS181. In some cases, the method comprises administering a liquid composition comprising a suspension of microparticles comprising DAS181.

In the above method, administration of the compound having sialidase activity leads to an improvement in the parameters resulting from the infection of the eye. In a preferred embodiment, the method leads to one or more of these parameters: a reduction of conjunctival congestion, a reduction of vascular dilatation, a reduction of edema in the tissue surrounding the eye, a reduction of hemorrhaging in and around the eye tissue, a reduction of hyperaemia (red eye), a reduction of chemosis (swelling of the conjunctiva), a reduction of epiphora (watering) in and around the eye, a reduction of pain in and around the eye, a reduction of the viral titer in the eye tissue, and an increase in visual acuity.

### DETAILED DESCRIPTION

In general, the present disclosure relates to methods for treating infections of the eye caused by a virus from the Picornavirdae family with compounds having sialidase activity. These compounds are described in U.S. Pat. Nos. 8,084,036 and 7,807,174 which are both hereby incorporated by reference in their entirety. The compounds having sialidase activity can remove sialic acid residues from the surface of cells and reduce infection by certain viruses that binding to sialic acid residues, e.g., coxsackievirus A24 variant.

In some embodiments, the severity of the infection in the eye is reduced with the treatment of the compounds. The reduction of the severity of the infection can be measured by the reduction of the symptoms which present with the infection. In some embodiments, treatment of an eye infection with the compounds can lead to a decrease in viral titer in the fluid within and around the eye and in the orbit cavity. The infection of the eye may occur independently of other infections within the patient having the eye infection. In some embodiments, the infection of the eye is an infection associated with a primary infection within the body of the patient having the eye infection.

The compounds of the present disclosure have sialidase activity. In some instances, the compounds having sialidase activity are a fusion protein in which the portion having sialidase activity is fused to a protein or protein fragment not having sialidase activity. In some instances, the portion having sialidase activity is fused to an anchoring domain. In some instances the anchoring domain is GAG. In some instances, the portion having sialidase activity is fused to a second portion that has a net positive charge at physiological pH.

The viruses causing the infections of the eye in the present disclosure refer to the viruses of the Picornavirdae family. The picornaviuses are divided among a number of genera including the Enterovirus genus. This genus is comprised of a number of viruses including enterovirus 70 (EV70) and coxsackievirus A24 variant (CVA24v).

DAS181 (SEQ ID NOs: 13 and 14) is a fusion protein compound comprising the catalytic domain of a sialidase (A. viscous) and an anchoring domain that is a human amphiregulin GAG-binding domain. In some instances of the present disclosure, DAS181 could be used to treat the infection of the eye resulting from a virus from the Picornavirdae family.

Unless defined otherwise, all technical and scientific terms used herein have the same meaning as commonly understood by one of ordinary skill in the art to which this disclosure belongs. Generally, the nomenclature used herein and the manufacture or laboratory procedures described below are well known and commonly employed in the art. Conventional methods are used for these procedures, such as those provided in the art and various general references. Where a term is provided in the singular, the inventors also contemplate the plural of that term. Where there are discrepancies in terms and definitions used in references that are incorporated by reference, the terms used in this application shall have the definitions given herein. As employed throughout the disclosure, the following terms, unless otherwise indicated, shall be understood to have the following meanings:

A “target cell” is any cell that can be infected by a pathogen causing AHC. The target cell can be selected from the group comprising the epithelial cell of the eye, cornea cell of the eye, conjunctival cell of the eye, and sclera cell of the eye.

A “domain that can anchor said at least one sialidase domain to the membrane of a target cell”, also called an “extracellular anchoring domain” or simply, “anchoring domain” refers to a moiety that can interact with a moiety that is at or on the exterior of a cell surface or is in close proximity to the surface of a cell. An extracellular anchoring domain can be reversibly or irreversibly linked to one or more moieties, such as, preferably, one or more sialidase domains, and thereby cause the one or more attached therapeutic moieties to be retained at or in close proximity to the exterior surface of a eukaryotic cell. Preferably, an extracellular anchoring domain interacts with at least one molecule on the surface of a target cell or at least one molecule found in close association with the surface of a target cell. For example, an extracellular anchoring domain can bind a molecule covalently or noncovalently associated with the cell membrane of a target cell, or can bind a molecule present in the extracellular matrix surrounding a target cell. An extracellular anchoring domain preferably is a peptide, polypeptide, or protein, and can also comprise any additional type of chemical entity, including one or more additional proteins, polypeptides, or peptides, a nucleic acid, peptide nucleic acid, nucleic acid analogue, nucleotide, nucleotide analogue, small organic molecule, polymer, lipids, steroid, fatty acid, carbohydrate, or a combination of any of these.

As used herein, a protein or peptide sequences is “substantially homologous” to a reference sequence when it is either identical to a reference sequence, or comprises one or more amino acid deletions, one or more additional amino acids, or more one or more conservative amino acid substitutions, and retains the same or essentially the same activity as the reference sequence. Conservative substitutions may be defined as exchanges within one of the following five groups:

I. Small, aliphatic, nonpolar or slightly polar residues: Ala, Ser, Thr, Pro, Gly

II. Polar, negatively charged residues and their amides: Asp, Asn, Glu, GIn

III. Polar, positively charged residues: His, Arg, Lys

IV. Large, aliphatic nonpolar residues: Met, Leu, Ile, Val, Cys

V. Large aromatic residues: Phe, Try, Trp

Within the foregoing groups, the following substitution are considered to be “highly conservative”: Asp/Glu, His/Arg/Lys, Phe/TyrlTrp, and Met/LeulIle/Val. Semi-conservative substitutions are defined to be exchanges between two of groups (I)-(1Y) above which are limited to supergroup (A), comprising (I), (II), and (III) above, or to supergroup (B), comprising (IV) and (V) above. In addition, where hydrophobic amino acids are specified in the application, they refer to the amino acids Ala, Gly, Pro, Met, Leu, Ile, Val, Cys, Phe, and Trp, whereas hydrophilic amino acids refer to Ser, Thr, Asp, Asn, Glu, GIn, His, Arg, Lys, and Tyr.

As used herein, the phrase “therapeutically effective amount” refers to the amounts of active compounds or their combination that elicit the biological or medicinal response that is being sought in a tissue, system, animal, individual or human by a researcher, veterinarian, medical doctor or other clinician, which includes one or more of the following:

(1) inhibiting the disease and its progression; for example, inhibiting a disease, condition or disorder in an individual who is experiencing or displaying the pathology or symptomatology of the disease, condition or disorder (i.e., arresting further development of the pathology and/or symptomatology) such as in the case of CVA24v or EV70 infection of the eye, a) ameliorating conjunctival congestion, reducing vascular dilatation within and surrounding the eye, reducing edema in the tissue surrounding the eye, reducing hemorrhaging in and around the eye tissue, reducing hyperaemia, reducing chemosis, reducing epiphora in and around the eye, reducing pain in and around the eye, reducing the viral titer in the eye tissue, and increasing the visual acuity of the infected eye, and

(2) ameliorating the disease; for example, ameliorating a disease, condition or disorder in an individual who is experiencing or displaying the pathology or symptomatology of the disease, condition or disorder (i.e., reversing the pathology and/or symptomatology) such as in the case of CVA24v or EV70 infection of the eye, a) ameliorating conjunctival congestion, b) reducing vascular dilatation within and surrounding the eye, c) reducing edema in the tissue surrounding the eye, d) reducing hemorrhaging in and around the eye tissue, e) reducing hyperaemia, f) reducing chemosis, g) reducing epiphora in and around the eye, h) reducing pain in and around the eye, i) reducing the viral titer in the eye tissue, j) increasing the visual acuity of the infected eye, and k) reducing the viral titer in the fluid in and around the infected eye tissue.

As used herein, the phrase “treating (including treatment)” includes one or more of the following:

(1) inhibiting the disease and its progression; for example, inhibiting a disease, condition or disorder in an individual who is experiencing or displaying the pathology or symptomatology of the disease, condition or disorder (i.e., arresting further development of the pathology and/or symptomatology) such as in the case of CVA24v or EV70 infection of the eye, a) ameliorating conjunctival congestion, b) reducing vascular dilatation within and surrounding the eye, c) reducing edema in the tissue surrounding the eye, d) reducing hemorrhaging in and around the eye tissue, e) reducing hyperaemia, f) reducing chemosis, g) reducing epiphora in and around the eye, h) reducing pain in and around the eye, i) reducing the viral titer in the eye tissue, j) increasing the visual acuity of the infected eye, and k) reducing the viral titer in the fluid in and around the infected eye tissue, and

(2) ameliorating the disease; for example, ameliorating a disease, condition or disorder in an individual who is experiencing or displaying the pathology or symptomatology of the disease, condition or disorder (i.e., reversing the pathology and/or symptomatology) such as in the case of CVA24v or EV70 infection of the eye, a) ameliorating conjunctival congestion, b) reducing vascular dilatation within and surrounding the eye, c) reducing edema in the tissue surrounding the eye, d) reducing hemorrhaging in and around the eye tissue, e) reducing hyperaemia, f) reducing chemosis, g) reducing epiphora in and around the eye, h) reducing pain in and around the eye, i) reducing the viral titer in the eye tissue, j) increasing the visual acuity of the infected eye, and k) reducing the viral titer in the fluid in and around the infected eye tissue.

A “sialidase” is an enzyme that can remove a sialic acid residue from a substrate molecule. The sialidases (N-acylneuraminosylglycohydrolases, EC 3.2.1.18) are a group of enzymes that hydrolytically remove sialic acid residues from sialo-glycoconjugates. Sialic acids are alpha-keto acids with 9-carbon backbones that are usually found at the outermost positions of the oligosaccharide chains that are attached to glycoproteins and glycolipids. One of the major types of sialic acids is N-acetylneuraminic acid (NeuSAc), which is the biosynthetic precursor for most of the other types. The substrate molecule can be, as nonlimiting examples, an oligosaccharide, a polysaccharide, a glycoprotein, a ganglioside, or a synthetic molecule. For example, a sialidase can cleave bonds having alpha (2,3)-Gal, alpha(2,6)-Gal, or alpha (2,8)-Gal linkages between a sialic acid residue and the remainder of a substrate molecule. A sialidase can also cleave any or all of the linkages between the sialic acid residue and the remainder of the substrate molecule. Two major linkages between NeuSAc and the penultimate galactose residues of carbohydrate side chains are found in nature, NeuSAc alpha (2,3)-Gal and NeuSAc alpha (2,6)-Gal. Both NeuSAc alpha (2,3)-Gal and NeuSAc alpha (2,6)-Gal molecules can be recognized by influenza viruses as the receptor, although human viruses seem to prefer NeuSAc alpha (2,6)-Gal, avian and equine viruses predominantly recognize NeuSAc alpha (2,3)Gal. A sialidase can be a naturally-occurring sialidase, an engineered sialidase (such as, but not limited to a sialidase whose amino acid sequence is based on the sequence of a naturally-occurring sialidase, including a sequence that is substantially homologous to the sequence of a naturally-occurring sialidase). As used herein, “sialidase” can also mean the active portion of a naturally-occurring sialidase, or a peptide or protein that comprises sequences based on the active portion of a naturally-occurring sialidase.

A “fusion protein” is a protein comprising amino acid sequences from at least two different sources. A fusion protein can comprise amino acid sequence that is derived from a naturally occurring protein or is substantially homologous to all or a portion of a naturally occurring protein, and in addition can comprise from one to a very large number of amino acids that are derived from or substantially homologous to all or a portion of a different naturally occurring protein. In the alternative, a fusion protein can comprise amino acid sequence that is derived from a naturally occurring protein or is substantially homologous to all or a portion of a naturally occurring protein, and in addition can comprise from one to a very large number of amino acids that are synthetic sequences.

A “sialidase catalytic domain protein” is a protein that comprises the catalytic domain of a sialidase, or an amino acid sequence that is substantially homologous to the catalytic domain of a sialidase, but does not comprises the entire amino acid sequence of the sialidase the catalytic domain is derived from, wherein the sialidase catalytic domain protein retains substantially the same activity as the intact sialidase the catalytic domain is derived from. A sialidase catalytic domain protein can comprise amino acid sequences that are not derived from a sialidase, but this is not required. A sialidase catalytic domain protein can comprise amino acid sequences that are derived from or substantially homologous to amino acid sequences of one or more other known proteins, or can comprise one or more amino acids that are not derived from or substantially homologous to amino acid sequences of other known proteins.

### I. Composition for Preventing or Treating Infection by a Pathogen

The present disclosure relates to compounds that include a peptide. The compounds include all or a catalytic portion of a sialidase. In some cases the compound includes at least one domain that can associate the sialidase or portion thereof with a eukaryotic cell. By “peptide or protein-based” compounds, it is meant that a compound that includes a portion having an amino acid framework, in which the amino acids are joined by peptide bonds. A peptide or protein-based compound can also have other chemical compounds or groups attached to the amino acid framework or backbone, including moieties that contribute to the anchoring activity of the anchoring domain, or moieties that contribute to the infection-preventing activity or the sialidase domain. For example, the protein-based therapeutics of the present disclosure can comprise compounds and molecules such as but not limited to: carbohydrates, fatty acids, lipids, steroids, nucleotides, nucleotide analogues, nucleic acid molecules, nucleic acid analogues, peptide nucleic acid molecules, small organic molecules, or even polymers. The protein-based therapeutics of the present disclosure can also comprise modified or non-naturally occurring amino acids. Non-amino acid portions of the compounds can serve any purpose, including but not limited to: facilitating the purification of the compound, improving the solubility or distribution of the compound (such as in a therapeutic formulation), linking domains of the compound or linking chemical moieties to the compound, contributing to the two dimensional or three-dimensional structure of the compound, increasing the overall size of the compound, increasing the stability of the compound, and contributing to the anchoring activity or therapeutic activity of the compound.

The peptide or protein-based compounds of the present disclosure can also include protein or peptide sequences in addition to those that comprise anchoring domains or sialidase domains. The additional protein sequences can serve any purpose, including but not limited to any of the purposes outlined above (facilitating the purification of the compound, improving the solubility or distribution or the compound, linking domains of the compound or linking chemical moieties to the compound, contributing to the two-dimensional or three-dimensional structure of the compound, increasing the overall size of the compound, increasing the stability of the compound, or contributing to the anchoring activity or therapeutic activity of the compound). Preferably any additional protein or amino acid sequences are part of a single polypeptide or protein chain that includes the sialidase domain or domains, but any feasible arrangement of protein sequences is within the scope of the present disclosure.

The anchoring domain and sialidase domain can be arranged in any appropriate way that allows the compound to bind at or near a target cell membrane such that the therapeutic sialidase can exhibit an extracellular activity that prevents or impedes infection of the target cell by a pathogen. The compound will preferably have at least one protein or peptide-based anchoring domain and at least one peptide or protein-based sialidase domain. In this case, the domains can be arranged linearly along the peptide backbone in any order. The anchoring domain can be N-terminal to the sialidse domain, or can be C-terminal to the sialidase domain.

It is also possible to have one or more sialidase domains flanked by at least one anchoring domain on each end. Alternatively, one or more anchoring domains can be flanked by at least one sialidase domain on each end. Chemical, or preferably, peptide, linkers can optionally be used to join some or all of the domains of a compound. It is also possible to have the domains in a nonlinear, branched arrangement. For example, the sialidase domain can be attached to a derivatized side chain of an amino acid that is part of a polypeptide chain that also includes, or is linked to, the anchoring domain.

A compound of the present disclosure can have more than one anchoring domain. In cases in which a compound has more than one anchoring domain, the anchoring domains can be the same or different. A compound of the present disclosure can have more than one sialidase domain. In cases in which a compound has more than one sialidase domain, the sialidase domains can be the same or different. Where a compound comprises multiple anchoring domains, the anchoring domains can be arranged in tandem (with or without linkers) or on alternate sides of other domains, such as sialidase domains. Where a compound comprises multiple sialidase domains, the sialidase domains can be arranged in tandem (with or without linkers) or on alternate sides of other domains, such as, but not limited to, anchoring domains.

A peptide or protein-based compound of the present disclosure can be made by any appropriate way, including purifying naturally occurring proteins, optionally proteolytically cleaving the proteins to obtain the desired functional domains, and conjugating the functional domains to other functional domains. Peptides can also be chemically synthesized, and optionally chemically conjugated to other peptides or chemical moieties. Preferably, however, a peptide or protein-based compound of the present disclosure is made by engineering a nucleic acid construct to encode at least one anchoring domain and at least one sialidase domain together (with or without nucleic acid linkers) in a continuous polypeptide. The nucleic acid constructs, preferably having appropriate expression sequences, can be transfected into prokaryotic or eukaryotic cells, and the therapeutic protein-based compound can be expressed by the cells and purified. Any desired chemical moieties can optionally be conjugated to the peptide or protein-based compound after purification. In some cases, cell lines can be chosen for expressing the protein-based therapeutic for their ability to perform desirable post-translational modifications (such as, but not limited to glycosylation).

A great variety of constructs can be designed and their protein products tested for desirable activities (such as, for example, binding activity of an anchoring domain or catalytic activity of a sialidase domain). The protein products of nucleic acid constructs can also be tested for their efficacy in preventing or impeding infection of a target cell by a pathogen. In vitro and in vivo tests for the infectivity of pathogens are known in the art, such as those described in the Examples for the infectivity of influenza virus.

### Anchoring Domain

As used herein, an “extracellular anchoring domain” or “anchoring domain” is any moiety that interact with an entity that is at or on the exterior surface of a target cell or is in close proximity to the exterior surface of a target cell. An anchoring domain serves to retain a compound of the present disclosure at or near the external surface of a target cell. An extracellular anchoring domain preferably binds 1) a molecule expressed on the surface of a target cell, or a moiety, domain, or epitope of a molecule expressed on the surface of a target cell, 2) a chemical entity attached to a molecule expressed on the surface of a target cell, or 3) a molecule of the extracellular matrix surrounding a target cell.

An anchoring domain is preferably a peptide or protein domain (including a modified or derivatized peptide or protein domain), or comprises a moiety coupled to a peptide or protein. A moiety coupled to a peptide or protein can be any type of molecule that can contribute to the interaction of the anchoring domain to an entity at or near the target cell surface, and is preferably an organic molecule, such as, for example, nucleic acid, peptide nucleic acid, nucleic acid analogue, nucleotide, nucleotide analogue, small organic molecule, polymer, lipids, steroid, fatty acid, carbohydrate, or any combination of any of these.

Target tissue or target cell type includes the sites in an animal or human body where a pathogen invades or amplifies. For example, a target cell can be an endothelial cell that can be infected by a pathogen. In some embodiments, the target cell can be an epithelial cell of the eye. In another example, a target cell can be a conjunctival cell of the eye. In another example, target cells can be cornea cells of the eye. In another example, a target cell is a sclera cell of the eye. A composition of the present disclosure can comprise an anchoring domain that can interact with a cell surface entity, for example, that is specific for the endothelial cell type. In another example, a target cell can be an epithelial cell and a composition of the present disclosure can bind an entity present on the cell surface of many epithelial cell types, or present in the extracellular matrix of different types of epithelial cells. In another embodiment, a composition of the present disclosure can comprise an anchoring domain that can bind a cell surface epitope, for example, that is specific for the conjunctiva cell type. In another embodiment, a composition of the present disclosure can comprise an anchoring domain that binds specifically to the cell surface entity of a cornea cell. In this case localized delivery of the composition can restrict its localization to the site of the epithelial cells, conjunctival cells, sclera cells and cornea cells of the eye that are targets of the pathogen.

A compound for treating infection by a pathogen can comprise an anchoring domain that can bind at or near the surface of a target cell. For example, heparin/sulfate, closely related to heparin, is a type of GAG that is ubiquitously present on cell membranes, including the surface of respiratory epithelium. Many proteins specifically bind to heparin/heparan sulfate, and the GAG-binding sequences in these proteins have been identified (Meyer, F A, King, M and Gelman, R A. (1975) Biochimica et BiophysicaActa 392: 223-232; Schauer, S. ed., pp 233. Sialic Acids Chemistry, Metabolism and Function. Springer-Verlag, 1982). For example, the GAG-binding sequences of human platelet factor 4 (PF4) (SEQ ID NO:2), human interleukin 8 (IL8) (SEQ ID NO:3), human antithrombin III (AT III) (SEQ ID NO:4), human apoprotein E (ApoE) (SEQ ID NO:5), human angio-associated migratory cell protein (AAMP) (SEQ ID NO:6), or human amphiregulin (SEQ ID NO:7) have been shown to have very high affinity (in the nanomolar range) towards heparin (Lee, M K and Lander, A D. (1991) Pro Natl Acad Sci USA 88:2768-2772; Goger, B, Halden, Y, Rek, A, Mosl, R, Pye, D. Gallagher, J and Kungl, A J. (2002) Biochem. 41:1640-1646; Witt, D P and Lander AD (1994) Curr Bio 4:394-400; Weisgraber, K H, Rail, S C, Mahley, R W, Milne, R W and Marcel, Y. (1986) J Bio Chem 261:2068-2076). These sequences, or other sequences that have been identified or are identified in the future as heparin/heparan sulfate binding sequences, or sequences substantially homologous to identified heparin/heparan sulfate binding sequences that have heparin/heparan sulfate binding activity, can be used as epithelium-anchoring-domains in compounds of the present disclosure that can be used.

### Sialidase Domain

A sialidase that can cleave more than one type of linkage between a sialic acid residue and the remainder of a substrate molecule, in particular, a sialidase that can cleave both α(2, 6)-Gal and α(2, 3)-Gal linkages can be used in the compounds of the disclosure. Sialidases include the large bacterial sialidases that can degrade the receptor sialic acids Neu5Ac alpha(2,6)-Gal and Neu5Ac alpha(2,3)-Gal. For example, the bacterial sialidase enzymes from Clostridium perfringens (Genbank Accession Number X87369), Actinomyces viscosus, Arthrobacter ureafaciens, or Micromonospora viridifaciens (Genbank Accession Number D01045) can be used. Sialidase domains of compounds of the present disclosure can comprise all or a portion of the amino acid sequence of a large bacterial sialidase or can comprise amino acid sequences that are substantially homologous to all or a portion of the amino acid sequence of a large bacterial sialidase. In one preferred embodiment, a sialidase domain comprises a sialidase encoded by Actinomyces viscosus, such as that of SEQ ID NO: 12, or a sialidase sequence substantially homologous to SEQ ID NO: 12. In yet another preferred embodiment, a sialidase domain comprises the catalytic domain of the Actinomyces viscosus sialidase extending from amino acids 274-666 of SEQ ID NO:12, or a substantially homologous sequence.

Additional sialidases include the human sialidases such as those encoded by the genes NEU2 (SEQ ID NO:8; Genbank Accession Number Y16535; Monti, E, Preti, Rossi, E., Ballabio, A and Borsani G. (1999) Genomics 57:137-143) and NEU4 (SEQ ID NO:9; Genbank Accession Number NM080741; Monti, E, Preti, A, Venerando, Band Borsani, G. (2002) Neurochem Res 27:646-663). Sialidase domains of compounds of the present disclosure can comprise all or a portion of the amino acid sequences of a sialidase or can comprise amino acid sequences that are substantially homologous to all or a portion of the amino acid sequences of a sialidase. Preferably, where a sialidase domain comprises a portion of the amino acid sequences of a naturally occurring sialidase, or sequences substantially homologous to a portion of the amino acid sequences of a naturally occurring sialidase, the portion comprises essentially the same activity as the intact sialidase. The present disclosure also includes sialidase catalytic domain proteins. As used herein a “sialidase catalytic domain protein” comprises a catalytic domain of a sialidase but does not comprise the entire amino acid sequence of the sialidase from which the catalytic domain is derived. A sialidase catalytic domain protein has sialidase activity. Preferably, a sialidase catalytic domain protein comprises at least 10%, at least 20%, at least 50%, at least 70% of the activity of the sialidase from which the catalytic domain sequence is derived. More preferably, a sialidase catalytic domain protein comprises at least 90% of the activity of the sialidase from which the catalytic domain sequence is derived.

A sialidase catalytic domain protein can include other amino acid sequences, such as but not limited to additional sialidase sequences, sequences derived from other proteins, or sequences that are not derived from sequences of naturally occurring proteins. Additional amino acid sequences can perform any of a number of functions, including contributing other activities to the catalytic domain protein, enhancing the expression, processing, folding, or stability of the sialidase catalytic domain protein, or even providing a desirable size or spacing of the protein.

A preferred sialidase catalytic domain protein is a protein that comprises the catalytic domain of the A. viscosus sialidase. Preferably, an A. viscosus sialidase catalytic domain protein comprises amino acids 270-666 of the A. viscosus sialidase sequence (SEQ ID NO:12). Preferably, an A. Viscosus sialidase catalytic domain protein comprises an amino acid sequence that begins at any of the amino acids from amino acid 270 to amino acid 290 of the A. viscosus sialidase sequence (SEQ ID NO: 12) and ends at any of the amino acids from amino acid 665 to amino acid 901 of said A. viscosus sialidase sequence (SEQ ID NO: 12), and lacks any A. viscosus sialidase protein sequence extending from amino acid 1 to amino acid 269. (As used herein “lacks any A. viscosus sialidase protein sequence extending from amino acid 1 to amino acid 269” means lacks any stretch of four or more consecutive amino acids as they appear in the designated protein or amino acid sequence.)

In some preferred embodiments, an A. viscosus sialidase catalytic domain protein comprises amino acids 274-681 of the A. viscosus sialidase sequence (SEQ ID NO: 12) and lacks other A. viscosus sialidase sequence. In some preferred embodiments, an A. viscosus sialidase catalytic domain protein comprises amino acids 274-666 of the A. viscosus sialidase sequence (SEQ ID NO: 12) and lacks any other A. viscosus sialidase sequence. In some preferred embodiments, an A. viscosus sialidase catalytic domain protein comprises amino acids 290-666 of the A. viscosus sialidase sequence (SEQ ID NO: 12) and lacks any other A. viscosus sialidase sequence. In yet other preferred embodiments, an A. viscosus sialidase catalytic domain protein comprises amino acids 290-681 of the A. viscosus sialidase sequence (SEQ ID NO: 12) and lacks any other A. viscosus sialidase sequence.

### Linkers

A compound of the present disclosure can optionally include one or more linkers that can join domains of the compound. Linkers can be used to provide optimal spacing or folding of the domains of a compound. The domains of a compound joined by linkers can be sialidase domains, anchoring domains, or any other domains or moieties of the compound that provide additional functions such as enhancing compound stability, facilitating purification, etc. A linker used to join domains of compounds of the present disclosure can be a chemical linker or an amino acid or peptide linker. Where a compound comprises more than one linker, the linkers can be the same or different. Where a compound comprises more than one linker, the linkers can be of the same or different lengths.

Many chemical linkers of various compositions, polarity, reactivity, length, flexibility, and cleavability are known in the art of organic chemistry. Preferred linkers of the present disclosure include amino acid or peptide linkers. Peptide linkers are well known in the art. Preferably linkers are between one and one hundred amino acids in length, and more preferably between one and thirty amino acids in length, although length is not a limitation in the linkers of the compounds of the present disclosure. Preferably linkers comprise amino acid sequences that do not interfere with the conformation and activity of peptides or proteins encoded by monomers of the present disclosure. Some preferred linkers of the present disclosure are those that include the amino acid glycine. For example, linkers having the sequence: (GGGGS (SEQ ID NO:10))n, where n is a whole number between 1 and 20, or more preferably between 1 and 12, can be used to link domains of therapeutic compounds of the present disclosure.

The present disclosure also includes nucleic acid molecules that encode protein-based compounds of the present disclosure that comprise at least one sialidase domain and at least one anchoring domain. The nucleic acid molecules can have codons optimized for expression in particular cell types, such as, for example E. coli or human cells. The nucleic acid molecules or the present disclosure that encode protein-based compounds of the present disclosure that comprise at least one sialidase domain and at least one anchoring domain can also comprise other nucleic acid sequences, including but not limited to sequences that enhance gene expression. The nucleic acid molecules can be in vectors, such as but not limited to expression vectors.

### Administration

The compounds of the disclosure can be administered by liquid or topical administration to cover the infected regions of the eye and the tissue surrounding the eye. In other instances, the compound can be administered periocularly. The periocular route can include subconjunctival, intracameral, subtenon, retrobulbar, intravitreal, posterior juxtascleral and peribulbar administration.

In one embodiment, the compound is administered to the surface of the cornea to treat the infection of the eye caused by viruses of the Picornavirdae family. In some embodiments, the compound is administered to the sclera. In other embodiments, the compound is administered to the conjunctiva. In some embodiments, the infection of the eye is AHC.

### Nucleic Acid Molecules

The present disclosure also comprises nucleic acid molecules that encode protein-based compounds of the present disclosure that comprise a catalytic domain of a sialidase. The nucleic acid molecules can have codons optimized for expression in particular cell types, such as, for example E. coli or human cells. The nucleic acid molecules or the present disclosure that encode protein-based compounds of the present disclosure that comprise at least one catalytic domain of a sialidase can also comprise other nucleic acid sequences, including but not limited to sequences that enhance gene expression. The nucleic acid molecules can be in vectors, such as but not limited to expression vectors.

### II. Pharmaceutical Compositions

The present disclosure includes compounds of the present disclosure formulated as pharmaceutical compositions. The pharmaceutical compositions comprise a pharmaceutically acceptable carrier prepared for storage and preferably subsequent administration, which have a pharmaceutically effective amount of the compound in a pharmaceutically acceptable carrier or diluent. Acceptable carriers or diluents for therapeutic use are well known in the pharmaceutical art, and are described, for example, in Remington's Pharmaceutical Sciences, 18th Ed., Mack Publishing Co., Easton, Pa. (1990)). Preservatives, stabilizers, dyes and even flavoring agents can be provided in the pharmaceutical composition. For example, sodium benzoate, sorbic acid and esters of p-hydroxybenzoic acid can be added as preservatives. In addition, antioxidants and suspending agents can be used.

The pharmaceutically effective amount of a test compound required as a dose will depend on the route of administration, the type of animal or patient being treated, and the physical characteristics of the specific animal under consideration. The dose can be tailored to achieve a desired effect, but will depend on such factors as weight, diet, concurrent medication and other factors which those skilled in the medical arts will recognize. In practicing the methods of the present disclosure, the pharmaceutical compositions can be used alone or in combination with one another, or in combination with other therapeutic or diagnostic agents. These products can be utilized in vivo, preferably in a mammalian patient, preferably in a human, or in vitro. In employing them in vivo, the pharmaceutical compositions can be administered to the patient in a variety of ways, including topically or perioculary. Periocular administration includes subconjunctival, subtenon, retrobulbar, intravitreal and peribulbar administration.

In preferred embodiments, these pharmaceutical compositions may be in the form of liquid or eyedrop form. In some cases the composition can be an aerosol or spray.

The formulations of this disclosure may be varied to include; (1) other acids and bases to adjust the pH; (2) other tonicity imparting agents such as sorbitol, glycerin and dextrose; (3) other antimicrobial preservatives such as other parahydroxy benzoic acid esters, sorbate, benzoate, propionate, chlorbutanol, phenyl ethyl alcohol, benzalkonium chloride, and mercurials; (4) other viscosity imparting agents such as sodium carboxymethylcellulose, microcrystalline cellulose, polyvinylpyrrolidone, polyvinyl alcohol and other gums; (5) suitable absorption enhancers; (6) stabilizing agents such as antioxidants, like bisulfite and ascorbate, metal chelating agents such as sodium edetate and drug solubility enhancers such as polyethylene glycols.

In some embodiments, the agent is administered as an ophthalmic composition. Accordingly, in some embodiments, the methods comprise administration of the agent and an ophthalmically acceptable carrier. In some embodiments, the ophthalmic composition is a liquid composition, semi-solid composition, insert, film, microparticles or nanoparticles.

In some embodiments, the ophthalmic composition is a liquid composition. In some embodiments, the ophthalmic composition is a semi-solid composition. In some embodiments, the ophthalmic composition is a topical composition. The topical compositions include, but are not limited to liquid and semi-solid compositions. In some embodiments, the ophthalmic composition is a topical composition. In some embodiments, the topical composition comprises aqueous solution, an aqueous suspension, an ointment or a gel. In some embodiments, the ophthalmic composition is topically applied to the front of the eye, under the upper eyelid, on the lower eyelid and in the cul-de-sac. In some embodiments, the ophthalmic composition is sterilized. The sterilization can be accomplished by known techniques like sterilizing filtration of the solution or by heating of the solution in the ampoule ready for use. The ophthalmic compositions of the disclosure can further contain pharmaceutical excipients suitable for the preparation of ophthalmic formulations. Examples of such excipients are preserving agents, buffering agents, chelating agents, antioxidant agents and salts for regulating the osmotic pressure.

As used herein, the term “ophthalmically acceptable carrier” refers to any material that can contain and release the agent and that is compatible with the eye. In some embodiments, the ophthalmically acceptable carrier is water or an aqueous solution or suspension, but also includes oils such as those used to make ointments and polymer matrices such as used in ocular inserts. In some embodiments, the composition may be an aqueous suspension comprising the agent. Liquid ophthalmic compositions, including both ointments and suspensions, may have a viscosity that is suited for the selected route of administration. In some embodiments, the ophthalmic composition has a viscosity in the range of from about 1,000 to about 30,000 centipoise.

In some embodiments, the liquid composition further comprises a polymer. These polymers may be used to improve the bioavailability, raise viscosity, or reduce drainage from the eye for a liquid formulation. In some embodiments, the polymers include, but are not limited to, those described in Wagh, et al., “Polymers used in ocular dosage form and drug delivery systems”, Asian J. Pharm., pages 12-17 (January 2008), which is incorporated herein by reference in its entirety. In some embodiments, the polymer is sodium hyaluronase, chitosan, a cyclodextrin (e.g., hydroxypropyl b-cyclodextrin), polygalactoronic acid, xyloglucan, xanthan gum, gellan gum, a thiomer, a poly(ortho ester) (e.g., as described in Einmahl, Adv. Drug. Deliv. Rev. 53:45-73 (2001), which is incorporated herein by reference in its entirety), or a tamarind seed polysaccharide (e.g., as described in Ghelardi, et al., Antimicrob. Agents Chemother. 48:3396-3401 (2004), which is incorporated herein by reference in its entirety).

In some embodiments, the ophthalmic compositions may further comprise one or more of surfactants, adjuvants, buffers, antioxidants, tonicity adjusters, preservatives (e.g., EDTA, BAK (benzalkonium chloride), sodium chlorite, sodium perborate, polyquaterium-1), thickeners or viscosity modifiers (e.g., carboxymethyl cellulose, hydroxymethyl cellulose, polyvinyl alcohol, polyethylene glycol, glycol 400, propylene glycol hydroxymethyl cellulose, hydroxpropyl-guar, hyaluronic acid, and hydroxypropyl cellulose) and the like. Additives in the formulation may include, but are not limited to, sodium chloride, sodium bicarbonate, sorbic acid, methyl paraben, propyl paraben, chlorhexidine, castor oil, and sodium perborate.

Aqueous ophthalmic compositions (solutions or suspensions) generally do not contain physiologically or ophthalmically harmful constituents. In some embodiments, purified or deionized water is used in the composition. The pH may be adjusted by adding any physiologically and ophthalmically acceptable pH adjusting acids, bases or buffers to within the range of about 5.0 to 8.5. Ophthalmically acceptable examples of acids include acetic, boric, citric, lactic, phosphoric, hydrochloric, and the like, and examples of bases include sodium hydroxide, sodium phosphate, sodium borate, sodium citrate, sodium acetate, sodium lactate, tromethamine, trishydroxymethylamino-methane, and the like. Salts and buffers include citrate/dextrose, sodium bicarbonate, ammonium chloride and mixtures of the aforementioned acids and bases.

In some embodiments, the osmotic pressure of the ophthalmic composition may be from about 10 milliosmolar (mOsM) to about 400 mOsM, or from 260 to about 340 mOsM. In some embodiments, the osmotic pressure can be adjusted by using appropriate amounts of physiologically and ophthalmically acceptable salts or excipients. In further embodiments, sodium chloride may be used to approximate physiologic fluid. In other embodiments, the composition comprises sodium chloride ranging from about 0.01% to about 1% by weight, or from about 0.05% to about 0.45% by weight, based on the total weight of the composition. Equivalent amounts of one or more salts made up of cations such as potassium, ammonium and the like and anions such as chloride, citrate, ascorbate, borate, phosphate, bicarbonate, sulfate, thiosulfate, bisulfate, sodium bisulfate, ammonium sulfate, and the like can also be used in addition to or instead of sodium chloride to achieve osmolalities within the above stated range. Similarly, a sugar such as mannitol, dextrose, sorbitol, glucose and the like can also be used to adjust osmolality.

In some embodiments, the methods involve forming or supplying a depot of the agent in contact with the external surface of the eye. A depot refers to a source of agent that is not rapidly removed by tears or other eye clearance mechanisms. This allows for continued, sustained high concentrations of agent be present in the fluid on the external surface of the eye by a single application. Without wishing to be bound by any theory, it is believed that absorption and penetration may be dependent on both the dissolved drug concentration and the contact duration of the external tissue with the drug containing fluid. As the drug is removed by clearance of the ocular fluid and/or absorption into the eye tissue, more drug is provided, e.g. dissolved, into the replenished ocular fluid from the depot. Accordingly, the use of a depot may more easily facilitate loading of the ocular tissue for more insoluble agents. In some embodiments, the depot can remain for up to eight hours or more. In some embodiments, the ophthalmic depot forms include, but are not limited to, aqueous polymeric suspensions, ointments, and solid inserts. In some embodiments, a semi-solid composition is a liquid formulation which increases in viscosity upon application to the eye, usually because of a polymer in the liquid formulation. This viscosity increase may be triggered by a change in temperature, pH, or electrolyte concentration. In some embodiments, the polymer include, but are not limited to, those described for semi-solid dosage forms in Wagh, et al., “Polymers used in ocular dosage form and drug delivery systems”, Asian J. Pharm., pages 12-17 (January 2008), which is incorporated herein by reference in its entirety. In some embodiments, the polymer is celluloseacetophthalate, polyacrylic acid, gellan gum, hyaluronase, chitosan, salts of alginic acid (e.g., sodium alginate), or a block copolymer of ethylene oxide and propylene oxide (e.g., Pluronic®, BASF; poloxamer). In some embodiment, the polyacrylic acid is crosslinked acrylic acid (e.g., Carbopol®). In some embodiments, the semi-solid composition comprises a mixture of carbopol and a block copolymer of ethylene oxide and propylene oxide; a mixture of methyl cellulose and hydroxyethyl cellulose; or a mixture of polyethylene glycol and a block copolymer of ethylene oxide and propylene oxide.

In some embodiments, the ophthalmic composition is an ointment or gel. In some embodiment, the ophthalmic composition is an oil-based delivery vehicle. In some embodiments, the composition comprises a petroleum or lanolin base to which is added the active ingredient, usually as 0.1 to 2%, and excipients. Common bases may include, but are not limited to, mineral oil, petrolatum and combinations thereof. In some embodiments, the ointment is applied as a ribbon onto the lower eyelid.

In some embodiments, the ophthalmic composition is an ophthalmic insert. In some embodiments, the ophthalmic insert is biologically inert, soft, bio-erodible, viscoelastic, stable to sterilization after exposure to therapeutic agents, resistant to infections from air borne bacteria, bio-erodible, biocompatible, and/or viscoelastic. In some embodiments, the insert comprises an ophthalmically acceptable matrix, e.g., a polymer matrix. The matrix is typically a polymer and the agent is generally dispersed therein or bonded to the polymer matrix. In some embodiments, the agent may slowly release from the matrix through dissolution or hydrolysis of the covalent bond. In some embodiments, the polymer is bioerodible (soluble) and the dissolution rate thereof can control the release rate of the agent dispersed therein. In another form, the polymer matrix is a biodegradable polymer that breaks down such as by hydrolysis to thereby release the agent bonded thereto or dispersed therein. In further embodiments, the matrix and agent can be surrounded with an additional polymeric coating to further control release. In some embodiments, the insert comprises a biodegradable polymer such as polycaprolactone (PCL), an ethylene/vinyl acetate copolymer (EVA), polyalkyl cyanoacrylate, polyurethane, a nylon, or poly (dl-lactide-co-glycolide) (PLGA), or a copolymer of any of these. In some embodiments, the agent is dispersed into the matrix material or dispersed amongst the monomer composition used to make the matrix material prior to polymerization. In some embodiments, the amount of agent is from about 0.1 to about 50%, or from about 2 to about 20%. In further embodiments, the biodegradable or bioerodible polymer matrix is used so that the spent insert does not have to be removed. As the biodegradable or bioerodible polymer is degraded or dissolved, the agent is released.

In further embodiments, the ophthalmic insert comprises a polymer, including, but are not limited to, those described in Wagh, et al., “Polymers used in ocular dosage form and drug delivery systems”, Asian J. Pharm., pages 12-17 (January 2008), which is incorporated herein by reference in its entirety. In some embodiments, the insert comprises a polymer selected from polyvinylpyrrolidone (PVP), an acrylate or methacrylate polymer or copolymer (e.g., Eudragit® family of polymers from Rohm or Degussa), hydroxymethyl cellulose, polyacrylic acid, poly(amidoamine) dendrimers, poly(dimethyl siloxane), polyethylene oxide, poly(lactide-co-glycolide), poly(2-hydroxyethylmethacrylate), poly(vinyl alcohol), or polypropylene fumarate). In some embodiments, the insert comprises Gelfoam® R. In some embodiments, the insert is a polyacrylic acid of 450 kDa-cysteine conjugante.

In some embodiments, the ophthalmic composition is an ophthalmic film. Polymers suitable for such films include, but are not limited to, those described in Wagh, et al., “Polymers used in ocular dosage form and drug delivery systems”, Asian J. Pharm., pages 12-17 (January 2008), In some embodiments, the film is a soft-contract lense, such as ones made from copolymers of N,N-diethylacrylamide and methacrylic acid crosslinked with ethyleneglycol dimethacrylate.

In some embodiments, the insert comprises a core comprising the agent and an outer tube. In some embodiments, the outer tube may be permeable, semi-permeable, or impermeable to the drug. In some embodiments, the drug core may include a polymer matrix which does not significantly affect the release rate of the drug. In some embodiments, the outer tube, the polymer matrix of the drug core, or both may be bioerodible. In some embodiments, the co-extruded product can be segmented into drug delivery devices. In some embodiments, the devices may be left uncoated so that their respective ends are open, or the devices may be coated with, for example, a layer that is permeable to the agent, semi-permeable to the agent, or bioerodible. In certain embodiments, the agent and at least one polymer are admixed in powder form. In some embodiments, the insert is formed by forwarding a polymeric material to a first extrusion device, forwarding an agent to a second extrusion device, co-extruding a mass including the polymeric material and the agent, and forming the mass into at least one co-extruded drug delivery device which comprises a core including the agent and an outer layer including the polymeric material. In certain embodiments, the agent forwarded to the second extrusion device is in admixture with at least one polymer. In certain embodiments, the agent and the at least one polymer are admixed in powder form. In certain embodiments, this act includes forwarding more than one drug to the second extrusion device. In certain embodiments, the polymeric material is one of impermeable, semi-permeable, or permeable to the agent. The polymeric material may be bioerodible and/or radiation curable. In latter instances, the insert may be irradiated.

In certain embodiments, the insert is in a tubular form, and may be segmented into a plurality of shorter products. In certain embodiments, the insert further comprises a coating of the plurality of shorter products with one or more layers including at least one of a layer that is permeable to the agent, a layer that is semi-permeable to the agent, and a layer that is bioerodible. The polymeric material may include any biocompatible polymer, such as polycaprolactone (PCL), an ethylene/vinyl acetate copolymer (EVA), polyalkyl cyanoacrylate, polyurethane, a nylon, or poly (dl-lactide-co-glycolide) (PLGA), or a copolymer of any of these.

In some embodiments, the insert comprises a therapeutically effective amount of at least one agent coated by or dispersed in a polymer matrix, wherein the agent is in granular or particulate form. In some embodiments, the agent is released from the formulation as drug from the granules dissolves into or within the matrix, diffuses through the matrix, and is released into the surrounding physiological fluid. In some embodiments, the rate of release is limited primarily by the rate of dissolution of the agent from the granules/particles into the matrix; the steps of diffusion through the matrix and dispersion into the surrounding fluid are primarily not release-rate-limiting. In certain embodiments, the polymer matrix is non-bioerodible, while in other embodiments it is bioerodible. Exemplary non-bioerodible polymer matrices can be formed from polyurethane, polysilicone, poly(ethylene-co-vinyl acetate) (EVA), polyvinyl alcohol, and derivatives and copolymers thereof. Exemplary bioerodible polymer matrices can be formed from polyanhydride, polylactic acid, polyglycolic acid, polyorthoester, polyalkylcyanoacrylate, and derivatives and copolymers thereof.

In some embodiments, the insert comprises a collagenous material. In some embodiments, the insert may be a soluble ophthalmic drug insert (SODI, e.g., a polymeric oval film that can be introduced in the upper conjuctival sac for drug delivery; an elliptical insert such as OCUSERT® (Pilocarpine ocular therapeutic system, developed by Alza Corporation) which is made of ethylene vinyl acetate; OCUFIT® (developed by Escalon Ophthalmics Inc., Skillman, N S), which is a rod shaped silicone elastomer; Lacrisert®, a rod shaped insert made of cellulose; New Ophthalmic Drug Delivery Systems (NODS), made of poly (vinyl alcohol); and the inserts described in Fabrizio, Advanced Drug Delivery Reviews 16: 95-106, 1998, which is incorporated herein by reference in its entirety. In further embodiments, the insert can be placed, depending on the location and the mechanism used to hold the insert in position, by either the patient or the doctor. In further embodiments, the insert comprises collagen, gelatin, or a polymer, wherein the polymer is selected from polycaprolactone (PCL), an ethylene/vinyl acetate copolymer (EVA), polyalkyl cyanoacralate, polyurethane, a nylon, poly(dl-lactide-co-glycolide) (PLGA), or a copolymer of any of the aforementioned. In some embodiments, the insert is implanted under the upper eyelid. In some embodiments, the insert is implanted in the posterior segment of the eye, in the chroidal space, or in the sclera. In some embodiments, the insert is implanted intravitreally or sub-retinally. In some embodiments, the insert is injected sub-retinally. Methods of administration and techniques for their preparation are set forth in Remington's Pharmaceutical Sciences, which is incorporated herein by reference in its entirety.

In other embodiments, the insert provides a sustained release of the agent to the vitreous of the eye. As used herein, “sustained release” means that the composition releases the agent over an extended period of time in a controlled fashion. In some embodiments, the insert releases the agent at a rate such that the aqueous agent concentration remains less than the vitreous agent concentration during the release. In some embodiments, the aqueous agent concentration is from about 0.002 mg/mL to about 0.01 mg/mL, or from about 0.01 mg/mL to about 0.05 mg/mL, or less than about 0.05 mg/mL. In some embodiments, the agent is released at a rate of about 1 mg/day to about 50 mg/day, or from about 1 mg/day to about 10 mg/day. In some embodiments, the insert further comprises an additional therapeutic agent, as detailed above, e.g., fluocinolone acetonide (such as that found in the ophthalmic insert Retisert®). In some embodiments, the ophthalmic composition comprises microspheres or nanoparticles. In some embodiment, the microspheres comprise gelatin. In some embodiments, the microspheres are injected to the posterior segment of the eye, in the chroidal space, in the sclera, intravitreally or sub-retinally. In some embodiments, the micospheres or nanoparticles comprises a polymer including, but not limited to, those described in Wagh, et al., “Polymers used in ocular dosage form and drug delivery systems”, Asian J. Pharm., pages 12-17 (January 2008), which is incorporated herein by reference in its entirety. In some embodiments, the polymer is chitosan, a polycarboxylic acid such as polyacrylic acid, albumin particles, hyaluronic acid esters, polyitaconic acid, poly(butyl)cyanoacrylate, polycaprolactone, poly(isobutyl)caprolactone, poly(lactic acid-co-glycolic acid), or poly(lactic acid). In some embodiments, the microspheres or nanoparticles comprise solid lipid particles.

In some embodiments, the ophthalmic composition comprises an ion-exchange resin. In some embodiments, the ion-exchange resin is an inorganic zeolite or synthetic organic resin. In some embodiments, the ion-exchange resin includes, but is not limited to, those described in Wagh, et al., “Polymers used in ocular dosage form and drug delivery systems”, Asian J. Pharm., pages 12-17 (January 2008), which is incorporated herein by reference in its entirety. In some embodiments, the ion-exhange resin is a partially neutralized polyacrylic acid.

In some embodiments, the ophthalmic composition is an aqueous polymeric suspension. In some embodiments, the agent or a polymeric suspending agent is suspended in an aqueous medium (e.g., having the properties as described above). In some embodiment, the agent is suspended. In some embodiments, the agent is in solution. In further embodiments, the suspending agent serves to provide stability to the suspension, to increase the residence time of the dosage form on the eye, or to enhance the sustained release of the drug in terms of both longer release times and a more uniform release curve. Examples of polymeric suspending agents include, but are not limited to, dextrans, polyethylene glycols, polyvinylpyrolidone, polysaccharide gels, Gelrite®, cellulosic polymers like hydroxypropyl methylcellulose, and carboxy-containing polymers such as polymers or copolymers of acrylic acid, as well as other polymeric demulcents. In some embodiments, the polymeric suspending agent is a water swellable, water insoluble polymer, especially a crosslinked carboxy-containing polymer. In some embodiments, the polymeric suspending agent comprises from at least about 90% to about 99.9%, or from about 95% to about 99.9%, by weight based on the total weight of monomers present, of one or more carboxy-containing monoethylenically unsaturated monomers. In some embodiments, the carboxy-containing monoethylenically unsaturated monomer includes acrylic acid, methacrylic acid, ethacrylic acid, methylacrylic acid (crotonic acid), cis-a-methylcrotonic acid (angelic acid), trans-a-methylcrotonic acid (tiglic acid), a-butylcrotonic acid, a-phenylacrylic acid, a-benzylacrylic acid, a-cyclohexylacrylic acid, phenylacrylic acid (cinnamic acid), coumaric acid (o-hydroxycinnamic acid), and umbellic acid (p-hydroxycoumaric acid). In some embodiments, the polymers may be crosslinked by a polyfunctional crosslinking agent (e.g., a difunctional crosslinking agent). In further embodiments, the amount of crosslinking should be sufficient to form insoluble polymer particles, but not so great as to unduly interfere with sustained release of the agent. In some embodiment, the polymers are only lightly crosslinked. In some embodiments, the crosslinking agent is contained in an amount of from about 0.01% to about 5%, or from about 0.1% to about 5.0%, or from about 0.2% to about 1%, based on the total weight of monomers present. In some embodiments, the crosslinking agents are nonpolyalkenyl polyether difunctional crosslinking monomers such as divinyl glycol, 2,3-dihydroxyhexa-1,5-diene, 2,5-dimethyl-1,5-hexadiene, divinylbenzene, N,N-diallylacrylamide, N,N-diallymethacrylamide; polyalkenyl polyether crosslinking agents containing two or more alkenyl ether groupings per molecule, e.g., alkenyl ether groupings containing terminal H₂C═C<groups, prepared by etherifying a polyhydric alcohol containing at least four carbon atoms and at least three hydroxyl groups with an alkenyl halide such as allyl bromide or the like, e.g., polyallyl sucrose, polyallyl pentaerythritol, or the like; diolefinic non-hydrophilic macromeric crosslinking agents having molecular weights of from about 400 to about 8,000, such as insoluble diacrylates and polyacrylates and methacrylates of diols and polyols, diisocyanate hydroxyalkyl acrylate or methacrylate reaction products of isocyanate terminated prepolymers derived from polyester diols, polyether diols or polysiloxane diols with hydroxyalkylmethacrylates, and the like.

In some embodiments, the crosslinked polymers may be made from a carboxy-containing monoethylenically unsaturated monomer or monomers as the sole monoethylenically unsaturated monomer present, together with a crosslinking agent or agents. In some embodiments, the polymers are ones in which up to about 40%, and preferably from about 0% to about 20% by weight, of the carboxy-containing monoethylenically unsaturated monomer or monomers has been replaced by one or more non-carboxyl-containing monoethylenically unsaturated monomer or monomers containing only physiologically and ophthalmically innocuous substituents, including acrylic and methacrylic acid esters such as methyl methacrylate, ethyl acrylate, butyl acrylate, 2-ethylhexylacrylate, octyl methacrylate, 2-hydroxyethylmethacrylate, 3-hydroxypropylacrylate, and the like, vinyl acetate, N-vinylpyrrolidone, and the like (see Mueller et al. U.S. Pat. No. 4,548,990, the entire contents of which are incorporated herein by reference, for a more extensive listing of such additional monoethylenically unsaturated monomers). In some embodiments, the polymers include polycarbophil (Noveon AA-1), Carbopol®, and DuraSite®. In some embodiments, the crosslinked polymers are prepared by suspension or emulsion polymerizing the monomers, using conventional free radical polymerization catalysts, to a dry particle size of not more than about 50 mm in equivalent spherical diameter. In some embodiments, the average dry particle size is from about 1 to about 30 mm, or from about 3 to about 20 mm in equivalent spherical diameter. In some embodiments, the polymer particles are obtained by mechanically milling larger polymer particles. In further embodiments, such polymers will have a molecular weight from about 250,000 to about 4,000,000, and from 3,000,000,000 to 4,000,000,000. In other embodiments, the particles of crosslinked polymer are monodisperse, meaning that they have a particle size distribution such that at least about 80%, about 90% or about 95%, of the particles fall within a mm band of major particle size distribution. In further embodiments, the monodisperse particle size means that there is no more than about 20%, about 10%, or about 5% particles of a size below 1 mm. In some embodiments, the aqueous polymeric suspension comprises from about 0.05 to about 1%, from about 0.1 to about 0.5%, or from about 0.1 to about 0.5%, of the agent and from about 0.1 to about 10%, from about 0.5 to about 6.5%, from about 0.5 to about 2.0%, from about 0.5% to about 1.2%, from about 0.6 to about 0.9%, or from about 0.6 to about 0.8% of a polymeric suspending agent. Although referred to in the singular, it should be understood that one or more species of polymeric suspending agent can be used with the total amount falling within the stated ranges. In one embodiment, the amount of insoluble lightly crosslinked polymer particles, the pH, and the osmotic pressure can be correlated with each other and with the degree of crosslinking to give a composition having a viscosity in the range of from about 500 to about 100,000 centipoise, and preferably from about 1,000 to about 30,000 or about 1,000 to about 10,000 centipoise, as measured at room temperature (about 25° C.) using a Brookfield Digital LVT Viscometer equipped with a number 25 spindle and a 13R small sample adapter at 12 rpm. In some embodiments, the viscosity is from about 10 to about 400 centipoise, from about 10 to about 200 centipoises or from about 10 to about 25 centipoise.

In some embodiments, the aqueous polymeric suspensions may be formulated so that they retain the same or substantially the same viscosity in the eye that they had prior to administration to the eye. In some embodiments, they may be formulated so that there is increased gelation upon contact with tear fluid. For instance, when a formulation containing DuraSite® or other similar polyacrylic acid-type polymer is administered to the eye at a pH of less than about 6.7, the polymer may swell upon contact with tear fluid since it has a higher pH (around 7). This gelation or increase in gelation may lead to entrapment of the suspended particles, thereby extending the residence time of the composition in the eye. In some embodiments, the agent is released slowly as the suspended particles dissolve over time. In some embodiments, this delivery route increases patient comfort and increased agent contact time with the eye tissues, thereby increasing the extent of drug absorption and duration of action of the formulation in the eye. The agents contained in these drug delivery systems will be released from the gels at rates that depend on such factors as the drug itself and its physical form, the extent of drug loading and the pH of the system, as well as on any drug delivery adjuvants, such as ion exchange resins compatible with the ocular surface, which may also be present.

### III. Method of Treating an Infection by a Pathogen

The method of the present disclosure includes: treating a subject that is infected with a pathogen or at risk of being infected with a pathogen with a pharmaceutical composition of the present disclosure that comprises a protein-based compound that comprises a sialidase activity. In some preferred embodiments the method includes applying a therapeutically effective amount of a pharmaceutical composition of the present disclosure to epithelial cells of a subject. The sialidase activity can be an isolated naturally occurring sialidase protein, or a recombinant protein substantially homologous to at least a portion of a naturally occurring sialidase. A preferred pharmaceutical composition comprises a sialidase with substantial homology to the A. viscosus sialidase (SEQ ID NO:12). The subject to be treated can be an animal or human subject. In yet another aspect, the method includes: treating a subject that is infected with a pathogen with a pharmaceutical composition of the present disclosure that comprises a protein-based compound that comprises a sialidase catalytic domain. In some preferred embodiments, the method includes applying a therapeutically effective amount of a pharmaceutical composition of the present disclosure to epithelial cells of a subject. The sialidase catalytic domain is preferably substantially homologous to the catalytic domain of a naturally occurring sialidase. A preferred pharmaceutical composition comprises a sialidase catalytic domain with substantial homology to amino acids 274-666 the A. viscosus sialidase (SEQ ID NO: 12). The subject to be treated can be an animal or human subject. In some cases the compound is DAS181.

In some embodiments, the pathogen is one of the following: a member of the Picornavirdae virus family. In one embodiment, the pathogen is a Picornavirdae virus in the genus Enterovirus. In another embodiment, the pathogen is a coxsackievirus. In one preferred embodiment, the virus is CVA24v. In another preferred embodiment, the virus is EV70.

In some preferred embodiments of the present disclosure, the pharmaceutical composition prevents infection by a member of the Picornavirdae virus family, and a therapeutically effective amount of the pharmaceutical composition is applied to ocular cells of a subject. In some instances, the ocular cells are the epithelial cells of the eye or surrounding the eye. In another instance, the ocular cells are conjunctiva cells around the eye. In another instance, the ocular cells are the cells of the cornea. In other embodiments, a therapeutically effective amount of the compound is applied within the orbit cavity. This can be done by the use of administration of a liquid formulation, for example, by the use of eyedrops, a spray or aerosol. Preferably, the liquid administration is performed from one to four times a day. Because coxsackieviruses or enteroviruse viruses primarily infect the eye and the tissue within the orbit cavity, removing the receptor sialic acid locally on the cells of the the tissue within the orbit cavity can interrupt infections. The sialidase can be delivered to the surface of the eye (i.e. the cornea) and the tissue within the orbit cavity as an eyedrop or as an aerosol or spray, and it can be used either in therapeutic mode during early stage of the eye infection (or other infection) or prophylaxis. Alternatively, it can be delivered to tissue within the eye including the sclera or the iris. Similarly, the sialidase can be delivered as an eyedrop, aerosol or spray to reduce infection by coxsackievirus and enterovirus. It can also be delivered as an eyedrop, aerosol or spray to prevent or reduce colonization by pathogenic bacteria, including Streptococcus pneumoniae, Mycoplasma pneumoniae, Haemophilus influenzae, Moraxella catarrhalis and Pseudomonas aeruginosa. The therapeutic compounds can optionally be adapted, by genetic or chemical engineering, or by pharmaceutical formulation, to improve their half-life or retention within the ocular cavity epithelium.

### Dosage

As will be readily apparent to one skilled in the art, the useful in vivo dosage to be administered and the particular mode of administration will vary depending upon the age, weight and type of patient being treated, the particular pharmaceutical composition employed, and the specific use for which the pharmaceutical composition is employed. The determination of effective dosage levels, that is the dose levels necessary to achieve the desired result, can be accomplished by one skilled in the art using routine methods as discussed above. In non-human animal studies, applications of the pharmaceutical compositions are commenced at higher dose levels, with the dosage being decreased until the desired effect is no longer achieved or adverse side effects are reduced or disappear. The dosage for a compound of the present disclosure can range broadly depending upon the desired affects, the therapeutic indication, route of administration and purity and activity of the compound. Typically, human clinical applications of products are commenced at lower dosage levels, with dosage level being increased until the desired effect is achieved. Alternatively, acceptable in vitro studies can be used to establish useful doses and routes of administration of the test compound. Typically, dosages can be between about 1 ng/kg and about 10 mg/kg, preferably between about 10 ng/kg and about 1 mg/kg, and more preferably between about 100 ng/kg and about 100 micrograms/kg.

The exact formulation, route of administration and dosage can be chosen by the individual physician in view of the patient's condition (see, Fingle et al., in The Pharmacological Basis of Therapeutics (1975)). It should be noted that the attending physician would know how to and when to terminate, interrupt or adjust administration due to toxicity, organ dysfunction or other adverse effects. Conversely, the attending physician would also know to adjust treatment to higher levels if the clinical response were not adequate. The magnitude of an administrated does in the management of the disorder of interest will vary with the severity of the condition to be treated and to the route of administration. The severity of the condition may, for example, be evaluated, in part, by standard prognostic evaluation methods. Further, the dose and perhaps dose frequency will also vary according to the age, body weight and response of the individual patient, including those for veterinary applications.

Thus, in accordance with the present disclosure, there is further provided a method of treating and a pharmaceutical composition for treating virus infection of the eye. The treatment involves administering to a patient in need of such treatment a pharmaceutical carrier and a therapeutically effective amount of any composition of the present disclosure, or a pharmaceutically acceptable salt thereof.

In one preferred regimen, appropriate dosages are administered to each patient by either eyedrop, spray, or by aerosol. It will be understood, however, that the specific dose level and frequency of dosage for any particular patient maybe varied and will depend upon a variety of factors including the activity of the specific salt or other form employed, the metabolic stability and length of action of that compound, the age of the patient, body weight of the patient, general health of the patient, sex of the patient, diet of the patient, mode and time of administration, rate of excretion, drug combination, the severity of the particular condition, and the host undergoing therapy.

### EXAMPLES

### Example 1

### Preparation of a Suspension of DAS181 Microparticles for Use in Treating Eye Infections

Purification of DAS181

DAS181 is a fusion protein containing the heparin (glycosaminoglycan, or GAG) binding domain from human amphiregulin fused via its N-terminus to the C-terminus of a catalytic domain of Actinomyces Viscosus (e.g., sequence of amino acids set forth in SEQ ID NO: 13 (no amino terminal methionine) and SEQ ID NO: 14 (including amino terminal methionine). The DAS181 protein used in the examples below was purified as described in Malakhov et al., Antimicrob. Agents Chemother., 1470-1479 (2006), which is incorporated in its entirety by reference herein. Briefly, the DNA fragment coding for DAS181 was cloned into the plasmid vector pTrc99a (Pharmacia) under the control of an IPTG (isopropyl-β-D-thiogalactopyranoside)-inducible promoter. The resulting construct was expressed in the BL21 strain of Escherichia Coli(E. Coli). The E. coli cells expressing the DAS181 protein were washed by diafiltration in a fermentation harvest wash step using Toyopearl buffer 1, UFP-500-E55 hollow fiber cartridge (GE Healthcare) and a Watson-Marlow peristaltic pump. The recombinant DAS181 protein was then purified in bulk from the cells as described in US 20050004020 and US 20080075708, which are incorporated in their entirety by reference herein.

Activity of DAS181

The sialidase activity of DAS181 was measured using the fluorogenic substrate 4-methylumbelliferyl-N-acetyl-α-D-neuraminic acid (4-MU-NANA; Sigma). One unit of sialidase is defined as the amount of enzyme that releases 10 nmol of MU from 4-MU-NANA in 10 minutes at 37° C. (50 mM CH₃COOH—NaOH buffer, pH 5.5) in a reaction that contains 20 nmol of 4-MU-NANA in a 0.2 ml volume (Potier et al., Anal. Biochem., 94:287-296, 1979). The specific activity of DAS181 was determined to be 1,300 U/mg protein (0.77 μg DAS181 protein per unit of activity).

Microparticle Preparation

The following ingredients were then combined to form DAS181 microparticles in a large scale batch process:
(a) 75 mg/ml Histidine, 0.107M citric acid, pH 5.0 and 1M Trehalose stock solutions were sterile filtered into and combined in an Excipient Bottle.
(b) The contents of the Excipient Bottle were added, with mixing, to a Compounding Vessel containing 125 mg/ml DAS181 protein prepared as described in Example 1.
(c) Isopropanol was sterile filtered into an Isopropanol Bag
(d) The content of the Isopropanol Bag was pumped into the Compounding Vessel while mixing vigorously to form the Feedstock Solution. The final composition of the Feedstock Solution was as follows: 70 mg/ml DAS181, 26% isopropanol, 9.8 mg/ml histidine, 9.8 mg/ml trehalose, 2.69 mg/ml citric acid, pH 5.0. The time between initiating the addition of isopropanol and starting the lyophilization cycle was between 90 minutes and 120 minutes
(e) Stainless Steel trays that had undergone depyrogenation were each filled with 950 g of the Feedstock Solution, using a metering pump
(f) The filled Stainless Steel trays were subjected to a Lyophilization Cycle as follows:
   a. the feedstock solution in the lyophilization trays were gasketed and placed in the lyophilizer shelves at 25° C. for 5 minutes;
   b. the temperature of the shelves was lowered to −55° C. at a ramp rate of −0.4° C./minute;
   c. the trays were held at −55° C. for between 60 and 180 minutes;
   d. primary drying was accomplished by setting the condenser to <−60° C., applying a vacuum of 125 mTorr with 250 mTorr dead band and increasing the temperature to −40° C. at a ramp rate of 0.125° C./minute and further to a temperature of −30° C. at 0.167° C./minute;
   e. the temperature was held at −30° C. for between 5000 and 6500 minutes;
   f. secondary drying was accomplished by increasing the temperature to 15° C. at a ramp rate of 0.5° C./minute, holding at 15° C. for 30 minutes, then further ramping up to a temperature of 30° C. at a ramp rate of 0.5° C./minute;
   g. the temperature was held at 30° C. for between 300 and 500 minutes; and
   h. the vacuum was released and the lyophilizer was backfilled with nitrogen to prevent oxidation of the microparticle formulations before transferring into bottles for bulk mixing and aliquoting the bulk powder for storage at ≦−15° C.

Physical Parameters:

The DAS181 dry powder microparticles prepared according to the above method have a mass median aerodynamic diameter (MMAD) of about 10 microns and a GSD of between 1 and 2.

### Suspension of Microparticles

To prepare 1 ml of a 100 mg DAS181/ml suspension, 125 mg of microparticles prepared as described were placed in a vial in a controlled RH environment (typically 10-30% RH). Next, 450 μL of PEG 300 was added to the vial and gently mixed with the microparticles. The mixture was held for 5 minutes to allow the microparticles to interact with the PEG 300. Next, 450 μL of water is added to the vial and the contents are gently mixed for 2-3 minutes or until a homogeneous suspension is achieved.

Injectability was measured using a NE-1010 syringe pump with a DPM-3 digital mount meter attached to the plunger rail. Standard lmL BD syringes are used with 27G×½ PrecisionGlide BD needles. Injectability values are reported in unit of lbs of force measured. Viscosity was measured using a Brookfield DV-1 Prime with a CPE-44PY cup and a CPE-40 cone spindle. Injection force of less than 50N is considered as injectable. The conversion unit of lbs to N is 1 lbs=4.4 N.

The above method produced suspensions with good injectability. Good results were obtained when the ratio of PEG 300 to water was: 50:50, 65:35 and 75:25. When PEG 200 was used, good results were obtained when the ratio of PEG 300 to water was 65:35 and 75:25.

In addition to polyethylene glycol (PEG 200, PEG 300, PEG 400, PEG 500, PEG 600), polysorbate 80, polysorbate 20 (Polyoxyethylene (20) sorbitan monooleate), propylene glycol, thioglycerol, tricaprylin, triolein, and versetamide are useful first media for adding to the protein microparticles.

The second media is water that can include salts, buffers, preservatives and other pharmaceutically acceptable excipients.

1-23. (canceled)

24. A method of treating an infection of the eye by a Picornavirdae virus, the method comprising administering to an eye of the patient a therapeutically effective amount of a compound having sialidase activity.

25. The method of claim 24 wherein the Picornaviridae virus is in the genus Enterovirus.

26. The method of claim 25 wherein the member of the genus Enterovirus is a coxsackievirus.

27. The method of claim 25 wherein the member of the genus Enterovirus is enterovirus type 70.

28. The method of claim 26 wherein the coxsackievirus is coxsackievirus A24 variant.

29. The method of claim 24 wherein the compound having sialidase activity comprises a portion of a sialidase having sialidase activity.

30. The method of claim 29 wherein the compound comprises a fusion protein wherein the fusion protein comprises at least a first portion comprising a portion of a sialidase having sialidase activity and the second portion binds to a glycosaminoglacan (GAG).

31. The method of 29 wherein the compound comprises a fusion protein wherein the fusion protein comprises at least a first portion comprising a portion of a sialidase having sialidase activity and the second portion has a net positive charge at physiological pH.

32. The method of claim 30 wherein the portion that binds to a GAG is selected from the group comprising: human platelet factor 4 (SEQ ID NO: 2), human interleukin 8 (SEQ ID NO: 3), human antithrombin III (SEQ ID NO: 4), human apoprotein E (SEQ ID NO: 5), human angio associated migratory protein (SEQ ID NO: 6), and human amphiregulin (SEQ ID NO: 7).

33. The method of claim 24 wherein the compound having sialidase activity is a bacterial sialidase.

34. The method of claim 32 wherein the bacterial sialidase is selected from a group comprising: Vibrio cholera, Arthrobacter ureafaciens, Clostridium perfringens, Actinomyces viscosus, and Micromonospora viridifaciens.

35. The method of claim 24 wherein the compound having sialidase activity is a human sialidase.

36. The method of claim 24 wherein the compound is administered to cells of the eye.

37. The method of claim 24 wherein the compound is administered to conjunctiva cells of the eye.

38. The method of claim 24 wherein the compound is administered to cornea cells of the eye.

39. The method of a claim 24 wherein the compound is administered in a topical liquid.

40. The method of claim 24 wherein the administration of the compound having sialidase activity causes one or more of:
a reduction of conjunctival congestion,
a reduction of vascular dilatation,
a reduction of edema in the tissue surrounding the eye,
a reduction of hemorrhaging in and around the eye tissue,
a reduction of hyperaemia (red eye),
a reduction of chemosis (swelling of the conjunctiva),
a reduction of epiphora (watering) in and around the eye,
a reduction of pain in and around the eye,
a reduction of the viral titer in the eye tissue, and
an increase in visual acuity.

41. The method of claim 24 wherein the infection of the eye is associated with a primary infection selected from the group comprising: an infection of the upper respiratory tract, an infection of the throat and the common cold.

42. The method claim 24 wherein the compound having sialidase activity is DAS181.

43. The method of claim 24 wherein the method comprising administering a liquid composition comprising a suspension of microparticles comprising DAS181.


SEQUENCE LISTING (14 sequences)

SEQ ID NO: 1 - protein, 58 aa, Bos taurus [sequence omitted]

SEQ ID NO: 2 - protein, 24 aa, Homo sapiens
NGRRICLDLQAPLYKKIIKKLLES

SEQ ID NO: 3 - protein, 27 aa, Homo sapiens
GRELCLDPKENWVQRVVEKFLKRAENS

SEQ ID NO: 4 - protein, 34 aa, Homo sapiens
QIHFFFAKLNCRLYRKANKSSKLVSANRLFGDKS

SEQ ID NO: 5 - protein, 34 aa, Homo sapiens
ELRVRLASHLRKLRKRLLRDADDLQKRLAVYQAG

SEQ ID NO: 6 - protein, 12 aa, Homo sapiens
RRLRRMESESES

SEQ ID NO: 7 - protein, 21 aa, Homo sapiens
KRKKKGGKNGKNRRNRKKKNP

SEQ ID NO: 8 - protein, 379 aa, Homo sapiens
MASLPVLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLNDRARSLVVPAYAYRKLHPIQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPQWLLYTHPTHSWQRADLGAYLNPRPPAPEAWSEPVLLAKGSCAYSDLQSMGTGPDGSPLFGCLYEANDYEEIVFLMFTLKQAFPAEYLPQ

SEQ ID NO: 9 - protein, 424 aa, Homo sapiens
LAGGSVRWGALHVLGTAALAEHRSMNPCPVHDAGTGTVFLFFIAVLGHTPEAVQIATGRNAARLCCVASRDAGLSWGSARDLTEEAIGGAVQDWATFAVGPGHGVQLPSGRLLVPAYTYRVDRLECFGKICRTSPHSFAFYSDDHGRTWRCGGLVPNLRSGECQLAAVDGGQAGSFLYCNARSPLGSRVQALSTDEGTSFLPAERVASLPETAWGCQGSIVGFPAPAPNRPRDDSWSVGPRSPLQPPLLGPGVHEPPEEAAVDPRGGQVPGGPFSRLQPRGDGPRQPGPRPGVSGDVGSWTLALPMPFAAPPQSPTWLLYSHPVGRRARLHMGIRLSQSPLDPRSWTEPWVIYEGPSGYSDLASIGPAPEGGLVFACLYESGARTSYDEISFCTFSLREVLENVPASPKPPNLGDKPRGCCWPS

SEQ ID NO: 10 - protein, 5 aa, Artificial Sequence
  Synthetic construct
GGGGS

SEQ ID NO: 11 - DNA, 2742 bp, Actinomyces viscosus
  nanH gene for sialidase
atgacatcgcatagtcctttctcccggaggcgcctgccggccctcctgggctccctgccactggccgccaccggcctgatcgccgccgcacccccggcgcacgccgtccccacgtctgacggcctggccgacgtcaccatcacgcaggtgaacgcgcccgcggacggcctctactccgtcggcgatgtcatgaccttcaacatcaccctgaccaacaccagcggcgaggcccactcctacgccccggcctcgacgaacctgtccgggaacgtctccaagtgccggtggcgcaacgtcccggccgggacgaccaagaccgactgcaccggcctggccacgcacacggtgaccgccgaggacctcaaggccggtggcttcaccccgcagatcgcctacgaggtcaaggccgtggagtacgccgggaaggccctgagcaccccggagacgatcaagggcgcgacgagcccagtcaaggccaactcgctgcgggtcgagtcgatcacgccgtcgtcgagccaggagaactacaagctgggcgacaccgtcagctacacggtgcgcgtgcgctcggtgtcggacaagacgatcaacgtcgccgccaccgaatcctccttcgacgacctgggccgccagtgccactggggcggcctcaagccgggcaagggcgccgtctacaactgcaagccgctcacccacacgatcacgcaagccgacgtcgacgccggccgctggacgccatcgatcaccctgacggccaccggaaccgacggcgccaccctccagacgctcaccgccaccggcaacccgatcaacgtcgtcggcgaccacccgcaggccacgcccgcaccggcgcccgacgcgagcacggagctgccggcctcaatgagccaggcccagcacctggccgccaacacggccaccgacaactaccgcatcccggcgataccaccgcccccaatggggacctgctcatctcctacgacgagcgcccgaaggacaacggcaacggcggcagcgacgacccccaacccgaaccacatcgtccagcgccgctccaccgacggcggcaagacctggtcggcgcccacctacatccaccagggcacggagaccggcaagaaggtcggctactccgacccgagctacgtcgtcgatcaccagacgggcacgatcttcaacttccacgtcaagtcctacgaccagggctggggcggctcgcgcggcggcaccgacccggagaaccggggcatcatccaggccgaggtgtcgacctccacggacaacggctggacctggacgcaccgcacgatcaccgcggacatcacgaaggacaagccgtggaccgcgcgtttcgcggcctcgggccagggcatccagattcagcacgggccccacgccgggcgcctggtgcagcagtacacgatcaggaccgccggcgggccggtgcaggccgtctcggtctactccgacgaccacgggaagacgtggcaggccggcacgccgatcgggaccggcatggatgagaacaaggtcgttgagctctccgacggctccctcatgctcaactcgcgcgcctcggatggctccggcttccgcaaggtggcccactccaccgacggtgggcagacctggagcgagccggtgtccgacaagaacctgcccgactcggtggacaacgcccagatcatccgagccttcccgaacgccgcgccggacgacccgcgcgccaaggtgctgctgctgagccactcaccgaacccgcggccgtggtgccgtgaccgcggcaccatctcgatgtcctgcgacgacggcgcctcctggacgaccagcaaggtcttccacgagcccttcgtcggatacacgacgatcgcggtgcagtccgacggcagcatcgggctgctcagcgaggacgcccacaacggcgccgactacggcggcatctggtaccgcaacttcacgatgaactggctcggcgagcagtgcggccagaagccggcggagccgagcccgggccgtcgccgacggcggcaccctcagcggcaccgacggagaagccggccccgtcggccgcgccgagcgctgagcccacgcaggcaccggcaccatcctccgcgcccgagccgagcgctgcgcccgagccgagcaggccccggcgccggagcccacgaccgctccgagcacggagcccacaccggctcctgcgcccagtccgcacctgagcagaccgatgggccgaccgctgcgcccgcaccggagacgtcctctgcaccggccgccgaaccgacgcaggccccgacggtggcgccttctgttgagcccacgcaggctccgggtgcgcagccgagctcagcacccaagccgggggcgacgggtcgggccccgtcggtggtgaacccgaaggcgaccggggcggcgacggagcctgggacgccgtcatcgagcgcgagcccggcaccgagccggaacgcggcgccgacgccgaagccgggcatggagcccgatgagattgatcggccgtctgacggcaccatggcgcagccgaccggtgcgccagcgcgccgagtgccgcgccgacgcaggcggcgaaggccggcagcaggctgtctcgcacgggaccaacgcgctgctgatcctgggccttgcgggtgtcgcggttgtcggcgggtacctgctgctgcgggctcgccgttcgaagaactgaacacgcgacgagccggtcatccggctctgagcactgactga

SEQ ID NO: 12 - protein, 913 aa, Actinomyces viscosus
  nanH sialidase
MTSHSPFSRRRLPALLGSLPLAATGLIAAAPPAHAVPTSDGLADVTITQVNAPADGLYSVGDVMTFNITLTNTSGEAHSYAPASTNLSGNVSKCRWRNVPAGTTKTDCTGLATHTVTAEDLKAGGFTPQIAYEVKAVEYAGKALSTPETIKGATSPVKANSLRVESITPSSSQENYKLGDTVSYTVRVRSVSDKTINVAATESSFDDLGRQCHWGGLKPGKGAVYNCKPLTHTITQADVDAGRWTPSITLTATGTDGATLQTLTATGNPINVVGDHPQATPAPAPDASTELPASMSQAQHLAANTATDNYRIPAIPPPPMGTCSSPTTSARRTTATAAATTPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGPVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWCRDRGTISMSCDDGASWTTSKVFHEPFVGYTTIAVQSDGSIGLLSEDAHNGADYGGIWYRNFTMNWLGEQCGQKPAEPSPGRRRRRHPQRHRRRSRPRRPRRALSPRRHRHHPPRPSRALRPSRAGPGAGAHDRSEHGAHTGSCAQSAPEQTDGPTAAPAPETSSAPAAEPTQAPTVAPSVEPTQAPGAQPSSAPKPGATGRAPSVVNPKATGAATEPGTPSSSASPAPSRNAAPTPKPGMEPDEIDRPSDGTMAQPTGAPARRVPRRRRRRRPAAGCLARDQRAADPGPCGCRGCRRVPAAAGSPFEELNTRRAGHPALSTD

SEQ ID NO: 13 - protein, 443 aa, Artificial Sequence
  Synthetic Construct
VKRKKKGGKNGKNRRNRKKKNPGDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPAEGADYGGIWYRNFTMNWLGEQCGQKPAE

SEQ ID NO: 14 - protein, 444 aa, Artificial Sequence
  Synthetic Construct
MVKRKKKGGKNGKNRRNRKKKNPGDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPAEGADYGGIWYRNFTMNWLGEQCGQKPAE